(19) United States
(12) Patent Application Publication
(10) Pub. No.: US 2004/0112391 A1
(43) Pub. Date: Jun. 17, 2004

(54) Mixed liquid foam human body immobilization pad

(76) Inventor: Patrick Lai, Taipei Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/320,446

(22) Filed: Dec. 17, 2002

(51) Int. Cl. A61B 19/00

(52) U.S. Cl. 128/869

(57) ABSTRACT

A mixed liquid foam type human body immobilization pad comprising a vesicle bag, a separating device, foaming agents, and an outer bag. The separating device divides the vesicle bag into two portions. A foaming agent B is contained in the first bag portion and a foaming agent A is contained in the second bag portion. The separating device, such as a clasp or clip is easily removed to allow the first bag portion and the second bag portion to join and the foaming agent A and foaming agent B can flow together and mix, resulting in a chemical reaction. When a patient is positioned on the vesicle bag a custom mold is formed which closely fits the contours of a portion of the patient's body. The close fit of the custom mold ensures a high degree of accuracy and fixation of the patient during therapy, treatment, and rehabilitation.

### FIELD OF THE INVENTION

The present invention relates to an immobilization pad, more particularly to a human body immobilization pad which contours to the shape of a human body.

### BACKGROUND OF THE INVENTION

Radiotherapy is used in the healthcare field as a way of killing abnormal cells (i.e., tumors) by utilizing machines emitting radiation. In the past Co-60 machines were utilized. Currently linear accelerator utilizing photon beams are popularly used worldwide. Regardless of the machine used, the human body should remain fixed during treatment as radiation is emitted from treatment machines to kill the tumor or cancer cell on a specified portion of the human body.

Treatment usually requires repeated radiotherapy sessions and the portions of the human body being treated must be accurately targeted each time. Otherwise, if not accurately targeted, normal cells in other portions of the human body may be damaged. Additionally, the treatment or therapy may occur over the span of several weeks or more. This accurate fixing, positioning, and re-positioning is not only critical to the efficacy of the treatment and health of the patient, but is currently extremely difficult to achieve utilizing conventional methods.

Currently, the accuracy and the degree of operation convenience provided by conventional posterior immobilization pads used today are not satisfactory. Some of the conventional types are general use molds, water-cured molds, vacuum fixation pads, and foaming agent pads.

Most general use molds are used for fixing the head and neck portions and only provide basic contours. A conventional mold is illustrated in FIG. 1. Because these conventional molds are not adaptable to different body shapes and sizes of various individuals, they fail to provide good posterior fixation.

The water-cured mold is tolerable for fixing a patient position but is very inconvenient in use. This type of mold is illustrated in FIG. 2.

The vacuum fixation pad is also limited not only because the thickness for achieving the required fixation often makes the production of anterior thermoplastic cast difficult but also because the materials of vacuum pads will cause more skin reaction to patients during radiotherapy. This type of vacuum fixation pad is illustrated in FIG. 3.

The foaming agent pad is created by utilizing custom molds made by a foaming agent. This type of foaming agent pad is illustrated in FIG. 4. Traditionally, a bottle 90 having a foaming agent A is poured into another bottle 91 having a foaming agent B, and then mixed. Next, the mixed foaming agents are poured into a bag body 92. The bag body 92 is then closed. The portion of the body to be treated is then pressed onto the bag body 92. After the foaming reaction is complete, a mold to the special portion of the human body is formed.

However, the conventional foaming agent pad has several disadvantages. For example, the storage and inventory of the two types of foaming agent is not convenient. Also, during a treatment procedure, it is not convenient for medical personal to open, and mix the two bottles of foaming agents and then pour the solution into the bag body.

Additionally, during opening, mixing, and pouring, the foaming agents and solution frequently leak or spill from the bottles. This leakage or spillage can contaminate the bodies of patients or medical personnel, and is therefore a safety concern when using two-part foaming agents.

Furthermore, medical person or healthcare staff are also susceptible to suffering from the risk of inhaling volatile organic vapors during use or operation.

Additionally, the pads are often used for patient positioning in rehabilitation, or fixation of an injured portion of a patient. Therefore, additional considerations must be made for the convenient and safe storage of the pads.

Therefore, there is need for an improved method of mold production by foaming agent for use in providing effective and accurate patient immobilization while at the same time being safe and convenient to produce, store, and use.

### SUMMARY OF THE INVENTION

To achieve these and other advantages and in order to overcome the disadvantages of the conventional method in accordance with the purpose of the invention as embodied and broadly described herein, the present invention provides a human body immobilization pad which contours to the shape of the human body.

As shown above, conventional methods for providing fixation of a human body during treatment are inadequate or ineffective. Additionally, the safety offered to medical personnel and patients using these conventional methods, is not adequate.

The present invention discloses a mixed liquid foam type human body immobilization pad comprising a vesicle bag, a separating device, foaming agents, and an outer bag. The separating device divides the vesicle bag into two portions. A foaming agent B is contained in the first bag portion and a foaming agent A is contained in the second bag portion. The separating device, such as a clasp, clamp or clip is easily removed to allow the first bag portion and the second bag portion to join and the foaming agent A and foaming agent B can flow together and mix, resulting in a chemical reaction. In an embodiment of the present invention the separating device is attached on the outside of the outer bag. However, the separating device may alternatively be attached to the inner bag before the inner bag is sealed in the outer bag. When a patient is positioned on the vesicle bag a custom mold is formed which closely fits the contours of a portion of the patient's body. The close fit of the custom mold ensures a high degree of accuracy and fixation of the patient during therapy, treatment, and rehabilitation.

Therefore, an object of the present invention is to provide a mixed liquid foam type human body immobilization pad that makes the production and use of custom molds for human body fixation, simple, convenient, and effective.

Another object of the present invention is to provide a mixed liquid foam type human body immobilization pad that makes the production and use of custom molds for human body fixation, safe and thus reduces the risk of patients or medical professions suffering contamination from chemicals during use.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a drawing illustrating a conventional general use mold;

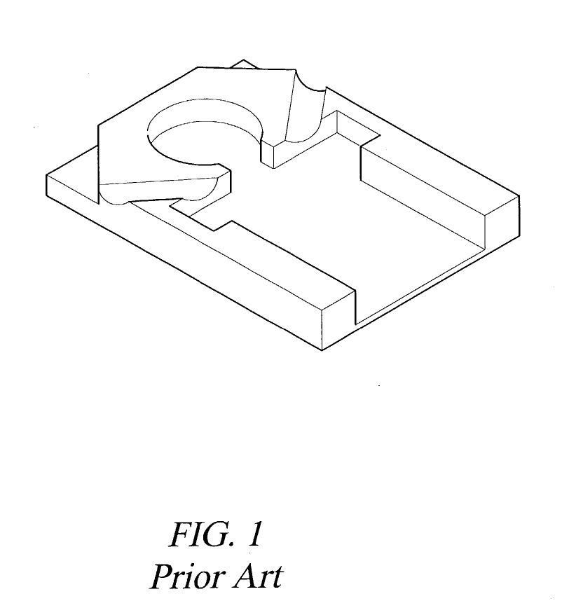

FIG. 2 is a drawing illustrating a conventional water-cured mold;

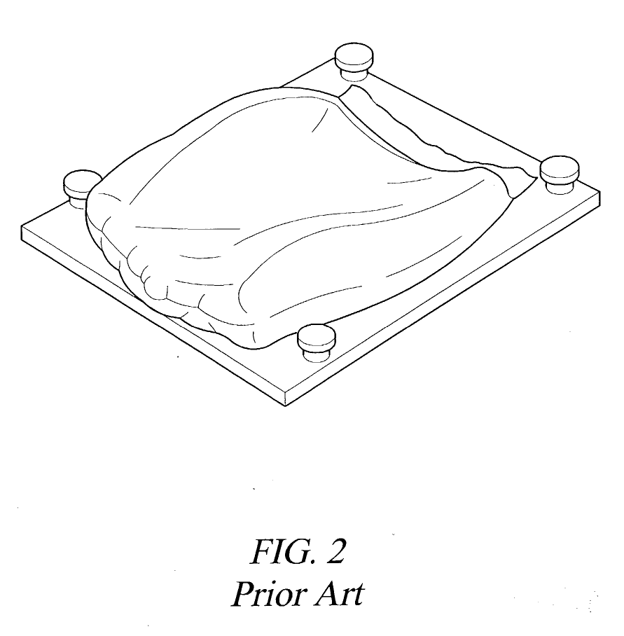

FIG. 3 is a drawing illustrating a conventional vacuum fixation pad;

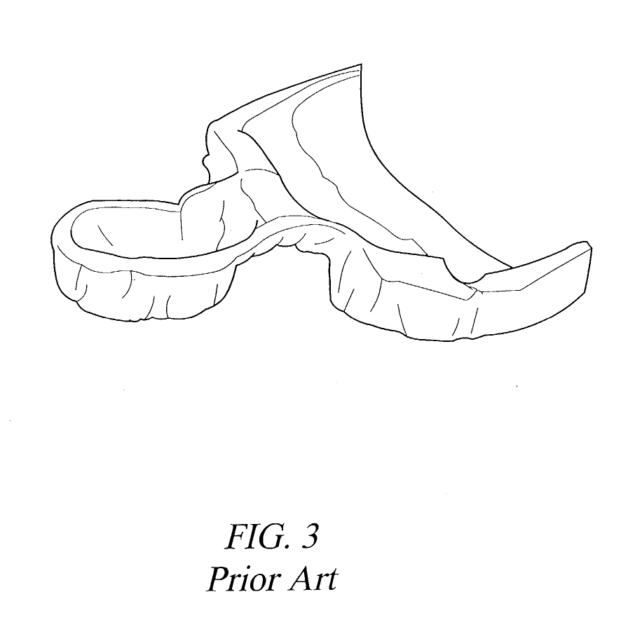

FIG. 4 is a drawing illustrating the foaming agent bottles and mixing bag for a conventional foaming agent pad;

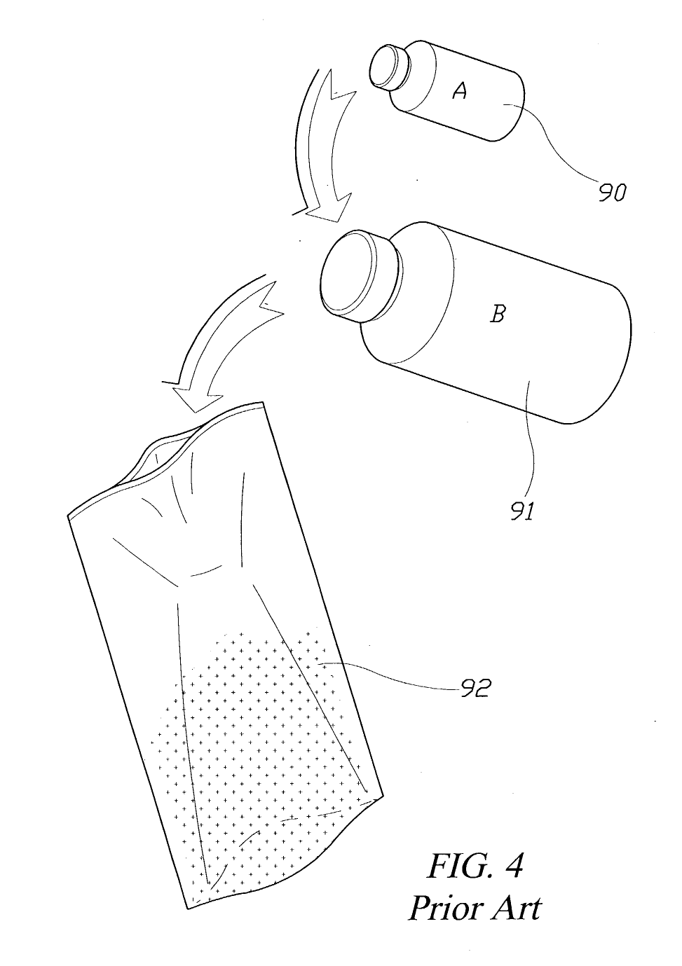

FIG. 5 is a drawing illustrating a mixed liquid foam type human body immobilization pad prior to use according to an embodiment of the present invention;

FIG. 6 is a drawing illustrating a mixed liquid foam type human body immobilization pad during mixing according to an embodiment of the present invention;

FIG. 7 is a drawing illustrating a mixed liquid foam type human body immobilization pad prior to use according to an embodiment of the present invention;

FIG. 8 is a side view drawing illustrating a mixed liquid foam type human body immobilization pad prior to use according to an embodiment of the present invention;

FIG. 9 is a drawing illustrating a mixed liquid foam type human body immobilization pad after forming according to an embodiment of the present invention; and

FIG. 10 is a drawing illustrating a mixed liquid foam type human body immobilization pad during use according to an embodiment of the present invention.

### DETAILED DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

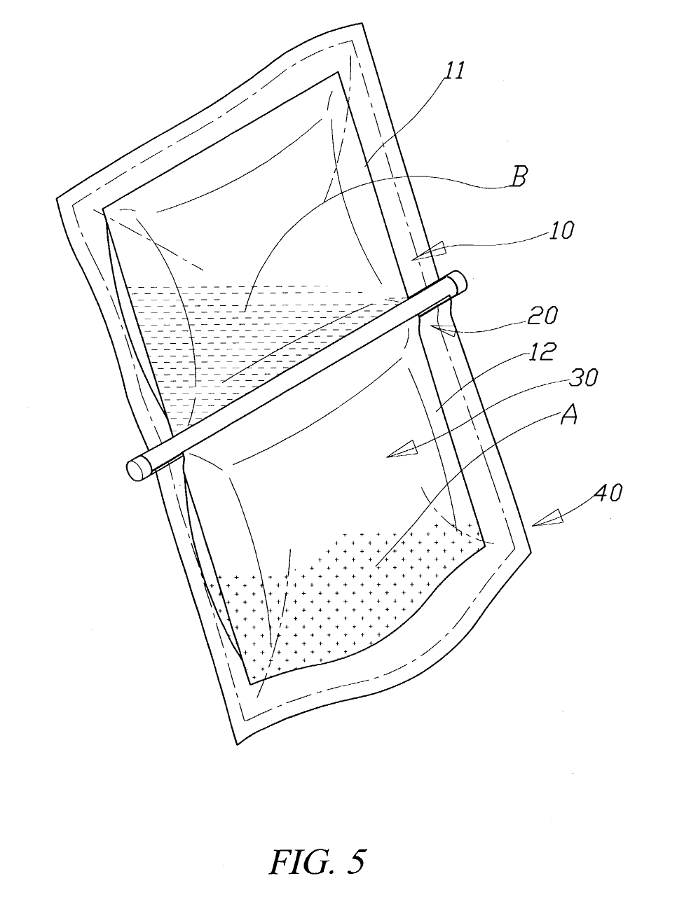

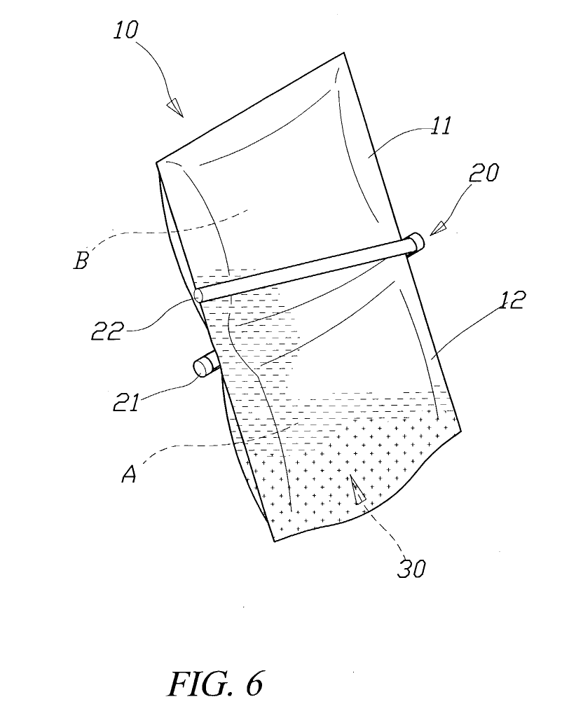

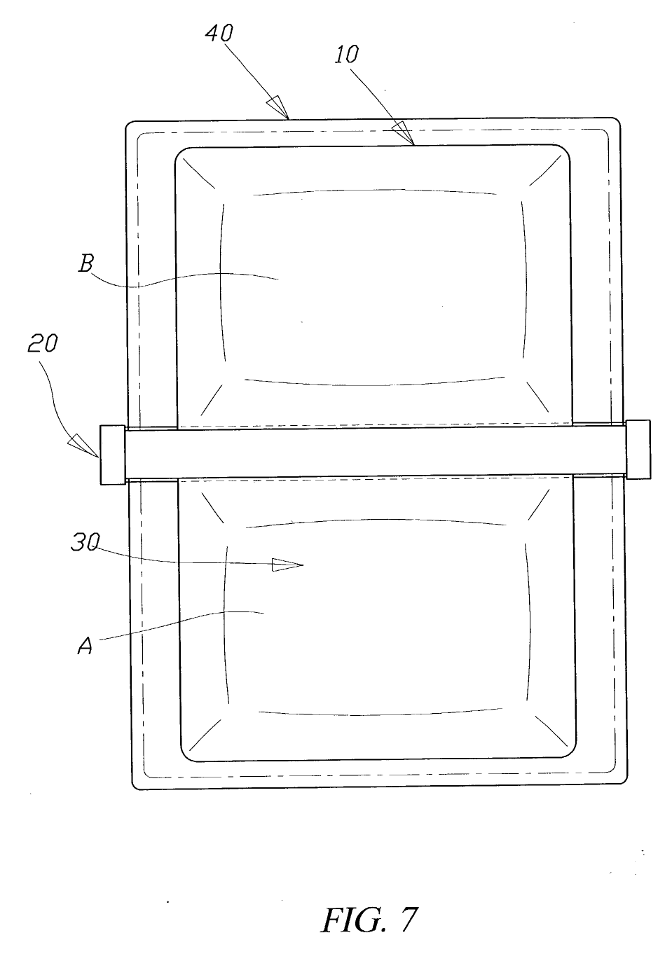

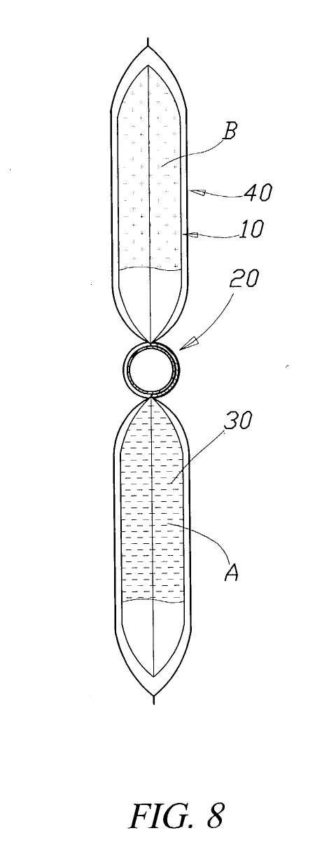

Refer to FIG. 5, which is a drawing illustrating a mixed liquid foam type human body immobilization pad prior to use according to an embodiment of the present invention, FIG. 6, which is a drawing illustrating a mixed liquid foam type human body immobilization pad during mixing according to an embodiment of the present invention, FIG. 7, which is a drawing illustrating a mixed liquid foam type human body immobilization pad prior to use according to an embodiment of the present invention, and FIG. 8, which is a side view drawing illustrating a mixed liquid foam type human body immobilization pad prior to use according to an embodiment of the present invention.

The mixed liquid foam type human body immobilization pad according to an embodiment of the present invention comprises a vesicle bag 10, a separating device 20, a foaming agent 30, and an outer bag 40. The vesicle bag 10 comprises a first bag portion 11 and a second bag portion 12. The separating device 20 separates the vesicle bag 10 into the first bag portion 11 and the second bag portion 12. A foaming agent B is contained in the first bag portion 11 and a foaming agent A is contained in the second bag portion 12.

In an embodiment of the present invention the separating device 20 is movably attached to the vesicle bag 10. The separating device 20 may be, for example, a clasp or clip type device or pin, so that the separating device 20 can be easily removed to allow the first bag portion 11 and the second bag portion 12 to join and the foaming agent A and foaming agent B can flow together and mix.

In an embodiment of the present invention the separating device 20 comprises a first clasp portion 21 and a second clasp portion 22. However, in other embodiments of the present invention, the separating device 20 is a pin, a tweezers type clasp, or any other type of device that efficiently separates the vesicle bag into two portions, so that once removed, the contents of the two portions are allowed to mix.

The foaming agents A and B are, for example, a reactant polyol blend and isocyanate as used in polyurethane. The outer bag 40 is constructed from a durable material such as, for example, an aluminum foil type material, that provides protection and encapsulation of the vesicle bag 10.

The mixed liquid foam type human body immobilization pad according to an embodiment of the present invention is produced by first securing the separating device 20 to the vesicle bag 10 or outer bag 40, thereby separating the vesicle bag 10 into a first portion 11 and second portion 12. For ease of manufacture, the vesicle bag may have openings at each end of the bag so that foaming agent A and foaming agent B may be introduced into the two portions of the vesicle bag 11, 12, after the separating device 20 divides the vesicle bag 10. After the foaming agents A and B are added to the two portions of the vesicle bag, these two openings may be sealed.

The mixed liquid foam type human body immobilization pad according to an embodiment of the present invention is efficiently formed for use by removing or opening the separating device 20, thereby allowing the foaming agent A and foaming agent B to mix.

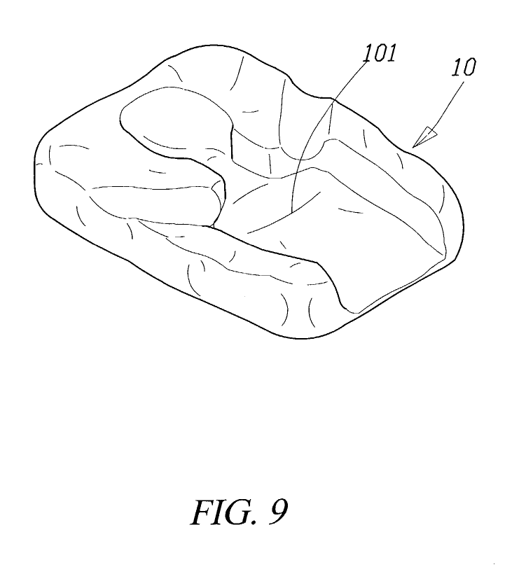

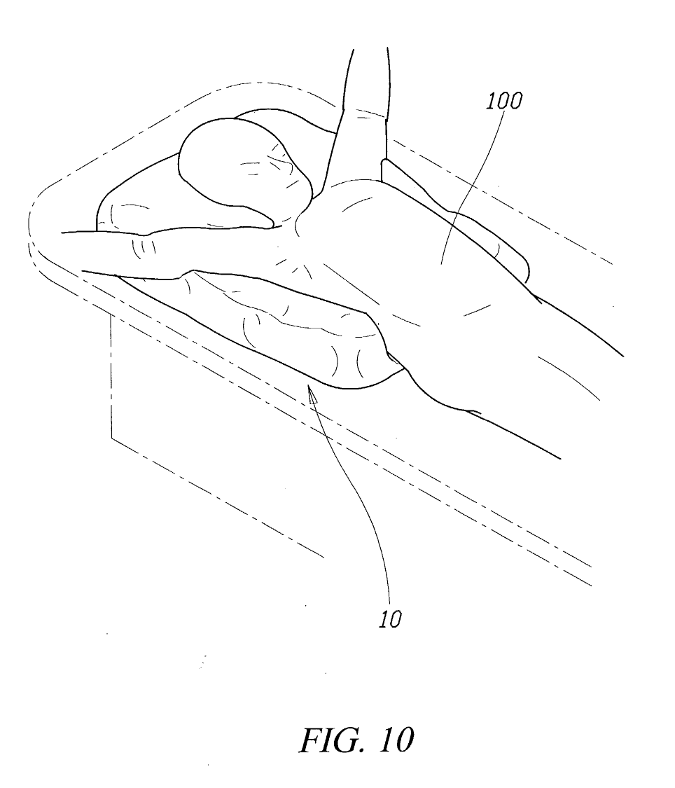

Refer to FIG. 9, which is a drawing illustrating a mixed liquid foam type human body immobilization pad after forming according to an embodiment of the present invention and FIG. 10, which is a drawing illustrating a mixed liquid foam type human body immobilization pad during use according to an embodiment of the present invention.

When the foaming agent A and foaming agent B mix a chemical reaction results whereby when a human body 100 is positioned on the vesicle bag, the foaming agents form a custom mold 101 closing fitting the contours of the human body 100.

The custom mold 101 closely fits to the contours of the human body 100 thereby ensuring a high degree of accuracy and fixation of the patient during radiotherapy, treatment, or rehabilitation.

As described above, the mixed liquid foam type human body immobilization pad of the present invention provides at least the following advantages over conventional devices:

Since it is unnecessary for medical personnel to open bottles and pour agents, leakages or spillages that can contaminate the bodies of patients or medical personnel is prevented. Also, accidental inhalation of chemical vapors is prevented due to the self-contained nature of the invention. Therefore, the safety of these individuals is ensured by utilization of the present invention, since the foaming agents are contained within the vesicle bag.

Similarly, since the foaming agents are already contained within the vesicle bag before use, storage and inventory requirements are eased.

Additionally, the production and use of the mixed liquid foam type human body immobilization pad of the present invention is simplified, making use and operation of the present invention quick, efficient, and effective. This also allows the production cost and operating expense to be reduced.

Furthermore, the accuracy of fixation of the present invention is superior as the custom mold closely fits the human body contours of the patient. Thereby, the therapy and treatment of medical problems is far more accurate and effective than tradition methods.

Therefore, the mixed liquid foam type human body immobilization pad of the present invention is safer, easier, quicker, more cost effective, accurate and effective than conventional pads.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. A mixed liquid foam type human body immobilization pad comprising:
a vesicle bag;
a separating device for separating the vesicle bag into a first bag portion and a second bag portion;
a first foaming agent contained in the first bag portion; and
a second foaming agent contained in the second bag portion;
whereby, when the separating device is removed, the first foaming agent and the second foaming agent are allowed to mix, resulting in a chemical reaction; and
whereby, the vesicle bag containing the mixed foaming agents becomes a custom mold closely fitting contours of a portion of a human body that contacts the custom mold.

2. The mixed liquid foam type human body immobilization pad of claim 1, whereby the separating device is a clasp.

3. The mixed liquid foam type human body immobilization pad of claim 2, whereby the clasp comprises a first clasp portion and a second clasp portion; and
whereby the first clasp portion and the second clasp portion attached together and surround the vesicle bag thus separating the vesicle bag into two portions.

4. The mixed liquid foam type human body immobilization pad of claim 1, wherein the vesicle bag is contained within an outer bag.

5. The mixed liquid foam type human body immobilization pad of claim 4, wherein the outer bag is comprised of aluminum foil.

6. The mixed liquid foam type human body immobilization pad of claim 1, wherein the foaming agents comprise polyol blend and isocyanate.

7. The mixed liquid foam type human body immobilization pad of claim 1, wherein the foaming agents are agents commonly used to produce polyurethane.

8. A mixed liquid foam type human body immobilization pad comprising:
a bag temporarily separated by a divider into a plurality of bag portions containing foaming agents;
whereby, when the divider is removed, the foaming agents mix and a custom mold is created closely fitting contours of a portion of a human body that contacts the custom mold.

9. The mixed liquid foam type human body immobilization pad of claim 8, whereby the divider is a clasp.

10. The mixed liquid foam type human body immobilization pad of claim 9, whereby the clasp comprises a first clasp portion and a second clasp portion; and
whereby the first clasp portion and the second clasp portion attached together and surround the vesicle bag thus separating the vesicle bag into a plurality of bag portions.

11. The mixed liquid foam type human body immobilization pad of claim 8, wherein the bag is contained within an outer bag.

12. The mixed liquid foam type human body immobilization pad of claim 11, wherein the outer bag is comprised of aluminum foil.

13. The mixed liquid foam type human body immobilization pad of claim 8, wherein the foaming agents comprise polyol blend and isocyanate.

14. The mixed liquid foam type human body immobilization pad of claim 8, wherein the foaming agents are agents commonly used to produce polyurethane.
